(19) United States
(12) Patent Application Publication
(10) Pub. No.: US 2025/0195563 A1
(43) Pub. Date: Jun. 19, 2025

(54) METHOD FOR GROWTH OF AKKERMANSIA MUCINIPHILA COMPRISING ADMINISTERING PLATINUM NANOPARTICLES

(71) Applicants: MR. WATERMAN CO., LTD., Tokyo (JP); NIHON TRIM CO., LTD., Osaka-shi, Osaka (JP)

(72) Inventor: Hitoshi HORI, Tokyo (JP)

(21) Appl. No.: 18/730,182

(22) PCT Filed: Jan. 19, 2023
(86) PCT No.: PCT/JP2023/001443

§ 371 Date: Jul. 18, 2024

(30) Foreign Application Priority Data
Jan. 19, 2022 (JP) 2022-006194

(51) Int. Cl. A61K 33/243; A61K 9/00; A61K 9/16; A61P 1/00

(52) U.S. Cl. CPC A61K 33/243; A61K 9/0095; A61K 9/16; A61P 1/00

(57) ABSTRACT

This invention is to provide the novel composition that grows Akkermansia muciniphila in the intestinal microflora. The composition for the growth of Akkermansia muciniphila containing platinum nanoparticles as an active ingredient.

### TECHNICAL FIELD

This invention relates to the composition for the growth of Akkermansia muciniphila.

### BACKGROUND ART

Akkermansia muciniphila, which is present in the intestinal microflora, contributes to the maintenance of the intestinal environment, and methods have been developed to increase its growth.

For example, Patent Document 1 describes the composition of an intestinal microflora improvement characterized in that it contains Gnetin C and/or its glycosides as active ingredients.

### CITATION LIST

### Patent Literature

Patent Document 1: International Publication No. 2020-071541

### SUMMARY OF INVENTION

### Technical Problem

This invention is to provide a novel composition that grows Akkermansia muciniphila in the intestinal microflora.

### Solution to Problem

This invention which solves the above problem, is the composition for the growth of Akkermansia muciniphila, containing platinum nanoparticles as an active ingredient.

According to this invention, Akkermansia muciniphila can be grown in the intestine.

In a preferred form of this invention, said platinum nanoparticles are dispersed in electrolyzed water.

According to this invention, Akkermansia muciniphila can be grown in the intestine.

In a preferred form of this invention, said platinum nanoparticles have an average particle size of 5 nm or less.

Also, in a preferred form of this invention, said platinum nanoparticles are contained at a concentration of 0.02 μg/mL or higher.

According to this invention, Akkermansia muciniphila can be grown in the intestine.

In a preferred form of this invention, the composition for the growth of Akkermansia muciniphila is a solid formulation and is dissolved in electrolyzed water to prepare the above composition.

According to this invention, Akkermansia muciniphila can be grown in the intestine.

### Advantageous Effect of Invention

According to this invention, the composition that grows Akkermansia muciniphila can be provided.

### BRIEF DESCRIPTION OF DRAWINGS

FIG. 1 shows the particle size distribution used to determine the average particle size of the platinum nanoparticles in this example.

### DESCRIPTION OF EMBODIMENTS

The composition for the growth of Akkermansia muciniphila contains platinum nanoparticles as an active ingredient.

According to this invention, Akkermansia muciniphila in the intestine (especially, in the large intestine) can be grown. The growth of Akkermansia muciniphila also strengthens the intestinal mucosal barrier and improves the intestinal environment.

In this invention, “platinum nanoparticles” refers to platinum particles with a diameter of 100 nm or less. Platinum nanoparticles can be produced by methods and equipment known in this field as appropriate.

Akkermansia muciniphila is found in the intestine, especially in the large intestine, and accounts for 1-4% of adult intestinal bacteria.

Akkermansia muciniphila secretes mucin, which thickens the mucus layer (mucin layer) in the intestinal mucosa, and the thickened mucus layer prevents the body from absorbing sugar, thus reducing sugar absorption, which is thought to have a slimming effect. In fact, it is known that Akkermansia muciniphila is absent or present only in very small amounts in the intestine of the persons who tend to be obese or have type II diabetes (adult onset type).

The mucus layer also physically prevents bacterial entry into the intestinal tissues by covering the intestinal epithelium, and the viscosity of the layer itself prevents bacterial adhesion to the epithelial cells.

By the way, when the intestinal mucosal barrier fails due to chronic inflammation in the intestine and bacteria invade the intestinal tissues, gaps are created in the tight junctions of the intestinal wall, resulting in enhanced intestinal permeability. This condition is called “leaky gut syndrome”.

When “leaky gut syndrome” develops and intestinal permeability enhances, viruses and bacteria leak from the intestine into the bloodstream, causing digestive disorders (such as irritable bowel syndrome), inflammatory bowel disease (such as ulcerative colitis and Crohn's disease), autoimmune diseases (such as rheumatoid arthritis and celiac disease), skin diseases (such as acne), neurodegenerative diseases (such as Alzheimer's disease dementia, Parkinson's disease, etc.), and various other diseases.

For example, it is known that when lipopolysaccharide (LPS) leaks into the blood from the intestine where intestinal leakage syndrome occurs and is transported to the brain, microglia are activated, and amyloid-β is produced and accumulated by the activated microglia, causing cognitive dysfunction.

It is also known that when LPS leaks from the intestine and is carried throughout the body in the bloodstream, the whole body enters a state of mild inflammation, which worsens insulin resistance and causes type II diabetes mellitus.

This invention is expected to prevent and/or improve intestinal leakage by increasing the amount of Akkermansia muciniphila in the intestinal microflora, thereby thickening the mucus layer of the intestinal mucosa and preventing and/or improving the formation of gaps in the tight junctions of the intestinal tract, thereby preventing and/or improving various diseases caused by the intestinal leakage syndrome. In addition, by thickening the mucus layer of the intestinal tract, an effect of improving the intestinal environment can be expected.

For example, a person whose blood LPS concentration exceeds a certain level has a high probability of having intestinal leakage syndrome. Such a person ingesting the composition of this invention can be expected to improve intestinal leakage and prevent or improve the various diseases mentioned above, by increasing the amount of Akkermansia muciniphila and thickening the mucus layer of the intestinal tract (in other words, strengthening the intestinal mucosal barrier).

In addition, by ingesting the composition of this invention, a person with a low amount of Akkermansia muciniphila in the intestinal microflora can increase the amount of Akkermansia muciniphila in the intestine and thicken the mucus layer (in other words, strengthen the intestinal mucosal barrier). This can be expected to improve obesity (slimming), prevent intestinal leakage, and prevent the various diseases mentioned above.

Akkermansia muciniphila in the intestinal microflora is known to stimulate enteroendocrine cells to secrete incretin, a digestive tract hormone. Incretin stimulates pancreatic islet of Langerhans beta cells to promote insulin secretion in a blood glucose level dependent manner.

Therefore, if Akkermansia muciniphila in the intestines is increased by ingesting the composition of this invention, the increased secretion of incretin and the resulting increased secretion of insulin can be expected to have a therapeutic or preventive effect on type II diabetes mellitus.

As mentioned above, LPS leaked from the intestines into the bloodstream is one of the causes of type II diabetes. However, if the composition of the present invention increases the amount of Akkermansia muciniphila in the intestine, thickens the mucus layer, and improves or prevents intestinal leakage, it can inhibit or prevent LPS leakage into the bloodstream. This can improve or prevent the inflammatory state of the whole body, which in turn improves insulin resistance and is expected to have a therapeutic or preventive effect on type II diabetes.

The composition of this invention is not restricted to any particular form, such as liquid, solid, and semi solid, etc.

When the composition of this invention is in liquid form, the dispersing medium should preferably be electrolyzed water. In other words, in the preferred form of this invention, the composition for the growth of Akkermansia muciniphila is the one in which platinum nanoparticles are dispersed in electrolyzed water.

Although it is known that platinum nanoparticles and electrolyzed water can remove active oxygen in the intestine, respectively, it is not known that especially Akkermansia muciniphila among the intestinal bacteria in the intestinal microflora, can be grown when these are taken simultaneously.

According to this invention, Akkermansia muciniphila in the intestines can be grown. The electrolyzed water can be made drinkable by known methods and equipment.

The pH of the electrolyzed water is preferably between 8 and 11, more preferably between 9 and 10, and even more preferably between 9.0 and 9.5.

The average particle diameter of the platinum nanoparticles should be 5 nm or less, 4.5 nm or less is more preferred, 4 nm or less is even more preferred, and 3 nm or less is even more preferred.

The average particle size should be 0.5 nm or larger, 0.75 nm or larger is more preferred, and 1.0 nm or larger is even more preferred.

According to this invention, Akkermansia muciniphila can be grown in the intestine.

In this invention, the average particle diameter is calculated by model numerical analysis using the small-angle X-ray scattering method. The detailed calculation method will be explained in the examples below.

When the invention is in liquid form, the concentration of platinum nanoparticles with respect to the entire composition should be 0.02 μg/mL or more, 0.024 μg/mL or more is more preferred, and 0.03 μg/mL or more is even more preferred. The concentration of platinum nanoparticles in the total composition should be 0.1 μg/mL or less, 0.096 μg/mL or less is more preferred, 0.09 μg/mL or less is even more preferred, 0.08 μg/mL or less is even more preferred, 0.076 μg/mL or less is even more preferred, 0.07 μg/mL or less is even more preferred, 0.06 μg/mL or less is even more preferred.

According to this invention, Akkermansia muciniphila can be grown in the intestine.

When this invention is in solid form (solid preparation), for example, it can be made into tablets or capsules. Tablets are preferred. Tablets can be taken orally as they are, or dissolved in water (preferably electrolyzed water) at the time of intake.

In the case of capsules, capsules containing platinum nanoparticles can be manufactured by any known method.

In the case of solid preparations, it is preferable that the content of platinum nanoparticles per tablet is 0.01 mg or more, more preferable that it is 0.016 mg or more, even more preferable that it is 0.02 mg or more, even more preferable that it is 0.024 mg or more, even more preferable that it is 0.03 mg or more.

The content of platinum nanoparticles per tablet should be 0.1 mg or less, 0.096 mg or less is more preferred, 0.09 mg or less is even more preferred, 0.08 mg or less is even more preferred, 0.076 mg or less is even more preferred, 0.07 mg or less is even more preferred, 0.06 mg or less is even more preferred.

A preferred form of this invention is a solid preparation (preferably in tablet form), for preparing the above composition by dissolving it in electrolyzed water.

According to this invention, the composition for the growth of Akkermansia muciniphila can be prepared in a liquid form by using electrolyzed water as a dispersing medium.

The composition of this invention for the growth of Akkermansia muciniphila can contain other known ingredients (such as taste enhancers, odor enhancers, pH adjusters, excipients, thickeners, flavoring agents, food dyes, sweeteners, dietary fiber, vitamins, preservatives, antioxidants, etc.) to the extent that the effect of this invention is not hindered.

The composition of this invention can be ingested to grow Akkermansia muciniphila in the intestines.

For example, when the composition of this invention for the growth of Akkermansia muciniphila is taken orally, in which platinum nanoparticles are dispersed in electrolyzed water, it is preferable that the daily intake of platinum nanoparticles is 10 μg or more, more preferable that the daily intake of platinum nanoparticles is 12 μg or more, even more preferable that the daily intake of platinum nanoparticles is 15 μg or more, even more preferable that the daily intake of platinum nanoparticles is 18 μg or more, even more preferable that the daily intake of platinum nanoparticles is 20 μg or more, even more preferable that the daily intake of platinum nanoparticles is 22 μg or more, even more preferable that the daily intake of platinum nanoparticles is 25 μg or more, even more preferable that the daily intake of platinum nanoparticles is 28 μg or more, and even more preferable that the daily intake of platinum nanoparticles is 30 μg or more.

According to this invention, Akkermansia muciniphila can be grown in the intestine.

The upper limit of the daily intake of platinum nanoparticles can be set at 50 μg or less, for example.

By promoting the growth of Akkermansia muciniphila as described above, the intestinal mucosal barrier can be strengthened, the intestinal environment can be improved, and leaky gut syndrome can be improved and/or prevented.

For example, if the dispersant is electrolyzed water and the concentration of platinum nanoparticles is 15 μg/500 mL, the above intake can be achieved by drinking 350 mL to 1 L or more per day.

As for the period of intake of the composition of this invention, it is preferable to take it for 4 or more consecutive weeks, more preferable to take it for 5 or more consecutive weeks, even more preferable to take it for 6 or more consecutive weeks, even more preferable to take it for 7 or more consecutive weeks, and even more preferable to take it for 8 or more consecutive weeks.

By ingesting this invention for the above periods of time, Akkermansia muciniphila can be grown in the intestine. There is no upper limit to the continuous intake period.

### EXAMPLES

In the following, this invention will be described more specifically with reference to examples, but the technical scope of the invention is not limited to the following examples.

A total of 13 randomly selected subjects (males and females in their 30s to 60s) were divided into a placebo group and a test group.

The placebo group was given two 500 mL bottled drinking water (tap water) per day for 8 consecutive weeks (1 L of water per day).

The test group was given two 500 mL bottled drinking water containing platinum nanoparticles dispersed in electrolyzed water (hereafter referred to as “electrolyzed nano platinum water”) per day for 8 consecutive weeks (1 L of water per day).

In this example, the average particle size of platinum nanoparticles in the electrolyzed nano platinum water was 2 nm, and the concentration of platinum nanoparticles per 500 mL PET bottle was 15 μg (i.e., the test group consumed 30 μg of platinum nanoparticles per day). The pH of the electrolyzed nano platinum water was 9.2. The method for measuring the average particle diameter of the platinum nanoparticles dispersed in the electrolyzed water in this example is shown below.

None of the subjects had any underlying diseases, and they lived their normal lives, except for drinking the above prescribed drinking water in the prescribed quantity.

### <Method of Measuring Average Particle Diameter of Platinum Nanoparticles>

A sample of platinum nanoparticles dispersed in milli-Q water was stirred and dispersed, filled into a glass capillary (1.5 mmφ), and measured. Milli-Q water was measured as a background.

Milli-Q water can be produced using Milli-Q (registered trademark) (Merck).

The average particle diameter was calculated using the small-angle X-ray scattering method as follows.

### (1) Equipment Used for Calculation

X-ray generator: RU-200 (rotating cathode type) manufactured by Rigaku Electric Co., Ltd. with a Cuka ray plane graphite incident monochromator as the X-ray source (output: 50 kV-200 mA)
Optics: Kratky camera manufactured by Rigaku Electric Co.
U-slit: width 70 mm, height 15 mm
Detector: Position sensitive proportional counter (PSPC), slit height 8.6 mm, integration time 60000 s, 1024 channels (88.03 channels/deg.), manufactured by Rigaku Electric Co.

### (2) Calculation Method

The intensity I(q) of the scattered wave due to the change in electron density of the scatterer (platinum particulate), which is regarded as a sphere, is expressed by the following equation (i).

$$I ( q ) = \vert Ω ( q ) \vert 2 ( q is the wavenumber vector ( = 4 π sin θ / λ ) ) \qquad ( i )$$

If the radius of the scatterer is R, the shape factor is expressed by the following equation (ii)

$$Ω ( q , R ) = 4 π R 3 / ( qR ) 3 \times ( sin ( qR ) - qR cos ( qR ) ) \qquad ( ii )$$

The particle size distribution P(R) is the Γ distribution of the following equation (iii).

$$[ Mathematical Expression 1 ]$$

$$P ( R ) = 1 / {Γ ( M ) [ M / {R}_{0} ]}^{M} \times exp ( - \frac{MR}{{R}_{0}} ) \times {R}^{- 1 + M} \qquad ( iii )$$

Here, in equation (iii), Γ(M) is the Γ function and R0 is the mean radius of the scatterer (mean particle diameter of platinum nanoparticles).

The normalized dispersion σ, where the dispersion is normalized by the mean value, is expressed as in Equation below (iv).

$$[ Mathematical Expression 2 ]$$

$$σ = \frac{\sqrt{〈 δ {R}^{2} 〉}}{{R}_{0}} = \frac{1}{\sqrt{M}} \qquad ( iv )$$

Here, in equation (iv), M is a parameter that determines the spread of the distribution: a large M gives a sharp distribution that is nearly symmetrical, similar to a Gaussian distribution, and a very broad distribution with no peak when M is less than 1.

The profiles after background processing were used for the analysis.

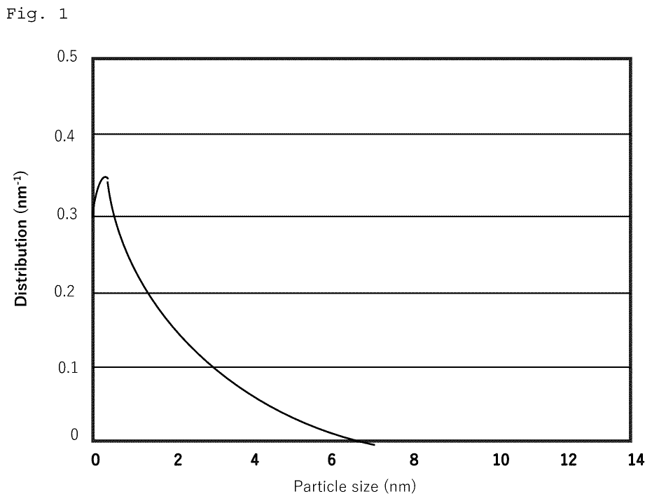

FIG. 1 shows the particle size distribution used to determine the average particle size of the platinum nanoparticles in this case.

The amount of Akkermansia muciniphila present in the feces (the amount of Akkermansia muciniphila present relative to the total amount of intestinal bacteria in the feces) and the concentration of LPS in the blood were measured before (day 0 of drinking) and after (8 weeks after drinking) the test.

The abundance of Akkermansia muciniphila in feces was determined by amplicon sequencing analysis targeting the 16s rDNA partial sequence according to the method of Takahashi et al. (Development of a prokaryotic universal primer for simultaneous analysis of Bacteria and Archaea using next-generation sequencing) by amplicon sequencing analysis targeting the 16s rDNA partial nucleotide sequence. Specifically, the following methods were used.

<Methods for Measuring the Abundance of Akkermansia muciniphila>

### <1> DNA Extraction

After suspending 100 mg of fecal sample collected from the subject in 4 M guanidium thiocyanate, 100 mM Tris-HCl (pH 9.0), 40 mM EDTA, FastPrep-2 5G (MP Biomedicals, USA) was used, and crushed with zirconia beads. The DNA concentration in each sample was adjusted to 10 ng/ml (measured by Nano Drop ND8000 (Themo Fisher Scientific, Inc.)).

### <2> Library Preparation

(2-1) Libraries were prepared by the touchdown PCR method using Pro341F as the forward primer and Pro805R as the reverse primer.

The reaction mixture (25 mL in total) contained 10 μg of genomic DNA generated in (1) above and 0.25 mM each of the above primers, and Mighty Amp for Real Time (Takara Bio) was used as the real-time PCR reagent.

PCR reaction conditions were as follows: after initial denaturation at 98° C. for 2 min, annealing at 65° C. to 55° C. for 15 s and elongation at 68° C. for 30 s, depending on the number of cycles, for 35 cycles. The annealing temperature was lowered by 1° C. for each cycle, and after reaching 55° C., the annealing temperature was kept constant at 55° C.

(2-2) PCR products were purified on a MultiScreen PCRu96 filter plate (Merck Millipore, USA) and analyzed using a Bioanalyzer DNA Chip 1000 Kit (Agilent Technologies, USA). The primer dimers were detected and the average molecular weight of each product was determined.

(2-3) Purified PCR products were quantified by real-time quantitative PCR (q-PCR) in a Rotor-Gene Q quantitative thermal cycler using the MightyAmp for Real Time (SYBR Plus).

The concentration the of library was determined by quantification by Miseq F as forward primer and Miseq R as reverse primer were added at 0.2 mM, and Phix control library (Illumina, USA) was serially diluted as standard.

PCR reaction conditions were as follows: initial denaturation at 98° C. for 2 min, followed by 30 cycles of denaturation at 98° C. for 10 sec, annealing at 60° C. for 15 sec, and extension at 68° C. for 30 sec.

<3> Amplicon Sequencing Analysis, Identification of Microorganisms and Calculation of the Amount of Akkermansia muciniphila Present

(3-1) Amplicon sequencing analysis was performed on the library prepared in <2> above using MiSeq Reagent Kit v3 (Illumina) as the sequencing kit and Miseq (Illumina) as the sequencer.

(3-2) Homology search by RDP Multi Classifier ver 2.11 and microorganism search by DB-BA13.0 were performed.

(3-3) Based on the search results in (4) above, the amount of Akkermansia muciniphila present in bacteria in the intestinal microflora was calculated.

The concentration of LPS in blood was measured by the turbidimetric time analysis method.

The results of each indicator are shown in Table 1 below.

In Table 1, 0% detection of Akkermansia muciniphila means that Akkermansia muciniphila was absent or below the detection limit.

A blood LPS concentration of 1.0 or less includes a LPS concentration below the detection limit.

TABLE 1

| | | | | Abundance of | | | | |
|---|---|---|---|---|---|---|---|---|
| | | | | Akkermansia | | | | |
| | | | | muciniphila (%) | | (%) | Blood LPS concentration | |
| Sub. | | | | Before | After | increase | Before | After |
| No. | sex | age | group | (0 days) | (8 weeks) | (times) | (0 days) | (8 weeks) |
| 1 | F | 53 | Test | 0.83 | 3.23 | 3.91 | 1.0 or less | 1.0 or less |
| 2 | M | 47 | Test | 0.64 | 2.11 | 3.31 | 1.0 or less | 1.0 or less |
| 3 | F | 39 | Test | 0.89 | 2.64 | 2.96 | 1.0 or less | 1.0 or less |
| 4 | F | 57 | Test | 0.00 | 0.14 | Detectability | 1.0 or less | 1.0 or less |
| 5 | F | 34 | Test | 4.21 | 0.32 | 0.08 | 1.0 or less | Not measuring |
| 6 | M | 49 | Placebo | 0.51 | 0.07 | 0.13 | 1.0 or less | 1.0 or less |
| 7 | F | 54 | Placebo | 2.00 | 0.29 | 0.15 | 1.0 or less | 1.3 |
| 8 | F | 59 | Placebo | 0.61 | 0.10 | 0.16 | 1.0 or less | 1.0 or less |
| 9 | M | 52 | Placebo | 0.00 | 0.00 | — | 1.0 or less | 1.0 or less |
| 10 | F | 48 | Placebo | 0.00 | 0.00 | — | 1.0 or less | 1.0 or less |
| 11 | M | 43 | Placebo | 0.00 | 0.00 | — | 1.0 or less | 1.1 |
| 12 | M | 30 | Placebo | 0.00 | 0.00 | — | 1.0 or less | 1.0 or less |
| 13 | F | 32 | Placebo | 7.09 | 2.80 | 0.40 | 1.0 or less | 1.0 or less |

As Table 1 shows, in the placebo group, the amount of Akkermansia muciniphila present was either unchanged at 0% (below the detection limit) (subject #9-12) or decreased (subject #6-8 and subject #13) after 8 weeks of testing.

In contrast, in the test group, the amount of Akkermansia muciniphila present increased after 8 weeks of testing.

These results indicate that this invention can increase the growth of Akkermansia muciniphila in the intestinal microflora.

In addition, using the amount of Akkermansia muciniphila in the intestinal microflora as an indicator, it was suggested that it is possible to selectively feed the composition of this invention to the persons with low amounts of Akkermansia muciniphila (e.g., the persons for whom the percentage of Akkermansia muciniphila in the bacteria in their feces is 1% or less).

As mentioned above, Akkermansia muciniphila is known to thicken the mucus layer in the intestinal mucosa.

From the above results, it is suggested that the mucus layer in the intestinal mucosa is thickened as a result of the growth of Akkermansia muciniphila in the large intestine by the ingestion of the composition of this invention. This also suggests that the intestinal mucosal barrier is strengthened and the intestinal environment is improved by ingesting the composition of this invention.

As represented in Table 1, there are two subjects (subject numbers 7 and 11) in the placebo group whose blood LPS concentration increased after the test compared to before the test.

As mentioned above, when intestinal leakage syndrome occurs, LPS leaks into the bloodstream, so the above two subjects with elevated blood LPS concentrations may be experiencing intestinal leakage.

In contrast, none of the subjects in the test group had elevated blood LPS levels after the 8-week study. In other words, no subject in the test group showed signs of intestinal leakage during the study period.

As described above, drinking platinum nano electrolyzed water causes the growth of Akkermansia muciniphila in the intestines.

The grown Akkermansia muciniphila thickens the mucus layer in the intestinal mucosa, and this suppressed the occurrence of intestinal leakage syndrome, as suggested by this example.

As described above, when intestinal leakage syndrome occurs, LPS in the blood is transported to various parts of the body, causing various diseases. It is suggested that the ingestion of the composition of this invention can prevent or treat various diseases caused by LPS.

It was also suggested that the LPS concentration in the blood could be used as an indicator to determine the selective ingestion of the composition of this invention for the persons with high LPS concentrations in the blood.

The above results suggest that the intake of the composition of this invention for the growth of Akkermansia muciniphila can strengthen the intestinal mucosal barrier and prevent and/or improve diseases such as Alzheimer's dementia and type II diabetes, which are considered to be one of the causes of intestinal leakage syndrome, as described above. In other words, it was suggested that the composition for the growth of Akkermansia muciniphila can be used for the prevention and/or treatment of diseases caused by the intestinal leakage syndrome.

### INDUSTRIAL APPLICABILITY

According to this invention, Akkermansia muciniphila in the intestinal microflora can be grown. This can also thicken the mucus layer of the intestinal mucosa and strengthen the intestinal mucosal barrier, thereby improving the intestinal environment.

1. A composition for the growth of Akkermansia muciniphila containing platinum nanoparticles as an active ingredient.

2. The composition according to claim 1, wherein said platinum nanoparticles are dispersed in electrolyzed water.

3. The composition for the growth of Akkermansia muciniphila according to claim 2, wherein the average particle size of said platinum nanoparticles is 5 nm or less.

4. The composition for the growth of Akkermansia muciniphila according to claim 2 or 3, comprising said platinum nanoparticles at the concentration of 0.02 μg/mL or more.

5. The composition for the growth of Akkermansia muciniphila according to claim 1, which is a solid formulation and is dissolved in electrolyzed water to prepare the composition as claimed in any one of claims 2 to 4.
